SEQUENCE LISTING (2 sequences)

SEQ ID NO: 1 - protein, 65 aa, Homo sapiens
SHLVKCAEKEKTFCVNGGECFMVKDLSNPSRYLCKCPNEFTGDRCQNYVMASFYKHLGIEFMEAE

SEQ ID NO: 2 - protein, 227 aa, Homo sapiens
SGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQK

(19) United States
(12) Patent Application Publication
(10) Pub. No.: US 2017/0035848 A1
(43) Pub. Date: Feb. 9, 2017

(54) TREATMENT OF NEPHROPATHY

(71) Applicant: UNIVERSITEIT ANTWERPEN, Antwerpen (BE)

(72) Inventors: Gilles DE KEULENAER, Boechout (BE); Vincent SEGERS, Hasselt (BE); Leni VANDEKERCKHOVE, Hemiksem (BE)

(21) Appl. No.: 15/303,778

(22) PCT Filed: Apr. 15, 2015
(86) PCT No.: PCT/EP2015/058117

§ 371 Date: Oct. 13, 2016

(30) Foreign Application Priority Data
Apr. 15, 2014 (EP) 14164741.2
Dec. 22, 2014 (EP) 14199547.2

(51) Int. Cl. A61K 38/18

(52) U.S. Cl. CPC A61K 38/1883

(57) ABSTRACT

The present invention relates to the treatment of kidney diseases, both acute and chronic. The invention in particular relates to the use of neuregulins for preventing, treating or delaying kidney diseases.

### FIELD OF THE INVENTION

Provided herein are compositions and methods for treating kidney diseases. More particularly the application relates to tools and methods for treating, preventing or delaying the development of kidney disease.

### BACKGROUND OF THE INVENTION

Kidneys perform several life-sustaining roles: they cleanse your blood by removing waste and excess fluid, maintain the balance of salt and minerals in your blood, and help regulate blood pressure. When the kidneys become damaged, waste products and fluid can build up in the body, causing swelling in your ankles, vomiting, weakness, poor sleep, and shortness of breath. If left untreated, diseased kidneys may eventually stop functioning completely. Loss of kidney function is a serious—and potentially fatal—condition.

Kidney disease, commonly denoted as nephropathy, can result from a variety of causes. It is generally characterized by failure to adequately maintain primary kidney functionality, accompanied by a reduction in glomerular filtration rate. Generally, nephropathy can be divided in acute kidney failure and chronic kidney disease.

Acute kidney failure—also called acute renal failure or acute kidney injury—develops rapidly over a few hours or a few days. Acute kidney injury has three main causes: (1) a sudden, serious drop in blood flow to the kidneys. Heavy blood loss, an injury, or a bad infection called sepsis can reduce blood flow to the kidneys. Not enough fluid in the body (dehydration) also can harm the kidneys. Also pregnancy complications such as eclampsia or pre-eclampsia may lead to acute kidney failure. (2) Damage from some medicines, poisons, or infections. Most people don't have any kidney problems from taking medicines. But people who have serious, long-term health problems are more likely than other people to have a kidney problem from medicines. Examples of medicines that can sometimes harm the kidneys include antibiotics, such as gentamicin and streptomycin; pain medicines, such as naproxen and ibuprofen; some blood sudden blockage that stops urine from flowing out of the kidneys. Kidney stones, a tumor, an injury, or an enlarged prostate gland can cause a blockage.

Kidney damage and decreased function that lasts longer than 3 months is called chronic kidney disease (CKD). Chronic kidney disease is particularly dangerous because you may not have any symptoms until considerable, often irreparable, kidney damage has occurred. Diabetes (types 1 and 2) and high blood pressure are the most common causes of CKD. Other causes are: (1) Immune system conditions such as lupus and chronic viral illnesses such as HIV/AIDS, hepatitis B, and hepatitis C; (2) Urinary tract infections within the kidneys themselves, called pyelonephritis, can lead to scarring as the infection heals. Multiple episodes can lead to kidney damage; (3) Inflammation in the tiny filters (glomeruli) within the kidneys; this can happen after strep infection and other conditions of unknown cause. (4) Polycystic kidney disease, in which fluid-filled cysts form in the kidneys over time. This is the most common form of inherited kidney disease. (5) Congenital defects, present at birth, are often the result of a urinary tract obstruction or malformation that affects the kidneys. One of the most common involves a valve-like mechanism between the bladder and urethra. These defects, sometimes found while a baby is still in the womb, can often be surgically repaired by a urologist. (6) Drugs and toxins, including long-term exposure to some medications and chemicals; overuse of NSAIDs (nonsteroidal anti-inflammatory drugs), such as ibuprofen and naproxen; and use of intravenous “street” drugs.

Diabetic nephropathy (DN) is the leading cause of chronic kidney failure resulting in end-stage renal disease (ESRD) and its prevalence is still increasing worldwide (1). Recent studies even show that the mortality rate among patients with type 1 and type 2 diabetes correlates with the presence and severity of kidney disease (2, 3). Absence of DN on the other hand, normalizes the risk of death in diabetic patients to the same level as that of the general population (4).

To date, DN treatment consists mainly of controlling hyperglycemia or pharmacologically inhibiting the renin-angiotensin-aldosteron system. However, these treatments do not improve kidney function and have not reduced the burden of ESRD (5, 6). Thus, new therapeutic approaches for DN are urgently needed. It is accordingly one of the objects of the present invention to overcome or ameliorate at least one of the disadvantages of the prior art, or to provide a useful alternative for the treatment of nephropathy, such as for instance diabetic nephropathy (nephrangiosclerosis), nephropathy caused by hypertension, nephropathy caused by vasculitis, lupus nephritis, nephropathy caused by glomerulonephritis, nephropathy caused by tubuloinsterstitial diseases, obstructive nephropathy, contrast induced nephropathy, glomerulonephritis, acute tubular necrosis (ATN), and acute interstitial nephritis (AIN).

### SUMMARY OF THE INVENTION

It has been surprisingly found that kidney damage can be prevented, delayed or treated in a mammal, by administration to said mammal of a neuregulin protein.

To date, neuregulins have not been shown to be involved in kidney function or pathology, let alone that there has been any indication of their potential in prophylactic or therapeutic treatment of nephropathy.

The present invention is in particular captured by any one or any combination of one or more of the below numbered aspects and embodiments (i) to (xvi).

(i) A neuregulin (NRG) protein for use in treating, preventing and/or delaying nephropathy in a mammal.

(ii) The NRG protein for use according to (i) or (ii), wherein said nephropathy is nephrosis or nephritis.

(iii) The NRG protein for use according to (i), wherein said nephropathy is chronic nephropathy.

(iv) The NRG protein for use according to (i), wherein said nephropathy is acute nephropathy.

(v) The NRG protein for use according to any of (i) to (iii), wherein said nephropathy is selected from the group consisting of diabetic nephropathy (nephrangiosclerosis), nephropathy caused by hypertension, nephropathy caused by vasculitis, lupus nephritis, nephropathy caused by glomerulonephritis, nephropathy caused by tubuloinsterstitial diseases, obstructive nephropathy, contrast induced nephropathy, toxic nephropathy, glomerulonephritis, acute tubular necrosis (ATN), and acute interstitial nephritis (AIN).

(vi) The NRG protein for use according to any of (i) to (iv), wherein said nephropathy is characterized by one or more of albuminuria, glomerulosclerosis, and/or renal fibrosis.

(vii) The NRG protein for use according to any of (i) to (vi), wherein said NRG protein suppresses collagen synthesis and/or FSP-1 synthesis, preferably in renal glomerular mesangial cells.

(viii) The NRG protein for use according to any of (i) to (vi), wherein administration of said NRG protein prevents a reduction of kidney function as determined by changes in proteinurea and/or albuminurea, GFR or S_{cr} (serum creatinine (mg/dL)).

(ix) The NRG protein for use according to any of (i) to (vi), wherein administration of said NRG protein increases GFR such as in conditions of acute or chronic kidney injury, such as in conditions of chronic kidney failure to delay hemodialysis.

(x) The NRG protein for use according to any of (i) to (vii), wherein said NRG protein is a neuregulin-1 (NRG-1) protein, a neuregulin-2 (NRG-2) protein, a neuregulin-3 (NRG-3) protein, a neuregulin-4 (NRG-4) protein, or mixtures thereof

(xi) The NRG protein for use according to any of (i) to (ix), wherein said NRG protein comprises an EGF-like domain.

(xii) The NRG protein for use according to any of (i) to (viii), wherein said NRG protein is an NRG1 type 1 protein.

(xiii) The NRG protein for use according to any of (i) to (x), wherein said NRG protein is to be administered daily.

(xiv) The NRG protein for use according to any of (i) to (xi), wherein said NRG protein is to be administered in a daily dose ranging from 0.01 to 100 μg/kg body weight.

(xv) The NRG protein for use according to any of (i) to (xii), wherein said mammal is a human.

(xvi) A nucleic acid encoding the NRG protein according to any of (i) to (xiii) for use in treating, preventing and/or delaying nephropathy in a mammal.

(xvii) A pharmaceutical composition comprising the NRG protein according to any of (i) to (xiii) or the nucleic acid according to (xiv) in an effective amount for use in treating, preventing and/or delaying nephropathy in a mammal.

The appended claims are also explicitly included in the description.

### BRIEF DESCRIPTION OF THE FIGURES

FIG. 1: Blood glucose in diabetic treated and untreated mice in comparison with non-diabetic littermates. Untreated diabetic mice display hyperglycemia (p<0.001) in comparison with control mice treated or untreated with NRG-1. Insulin treatment of diabetic animals results in normal blood glucose concentrations. NRG-1 has no effect on glycemia. * versus contr; # versus contr+NRG-1; $ versus Contr+INS.

FIG. 2: Urinary markers for kidney function. A, Microalbuminuria is induced in the STZ-treated animals (p<0.05), 14 weeks after induction of diabetes. Both insulin and NRG-1 treatment completely prevented the increase in urinary albumin (p<0.01, p<0.05 respectively). B, Urinary NGAL concentration was significantly higher in the diabetic animals in comparison with control animals (p<0.001). Insulin treatment prevented this upregulation (p<0.001), while there is a non-significant trend towards a decrease in urinary NGAL in the NRG-1 treated diabetic group.

FIG. 3: A, Glomerulosclerosis was absent in non-diabetic control animals in contradiction to STZ-treated mice (p<0.001) as shown by Masson's trichrome staining. Prevention of renal glomerular scarring is established by treating the animals with insulin or NRG-1 (p<0.001). Representative Masson's trichrome stained glomeruli of each group are shown, in which green staining indicates connective tissue. B, Presence of ErbB4 receptors within the renal glomeruli. Brown staining in the representative glomerulus indicates ErbB4 in comparison with the blank staining.

FIG. 4: Phosphorylation of Akt and Erk in renal glomerular mesangial cells treated with NRG-1.

FIG. 5: Upregulation of fibrosis markers in diabetic mice was prevented by NRG-1 treatment. A, There was a significant increase in mRNA expression of FSP-1 in the kidneys of untreated diabetic animals (p<0.001), which was prevented by NRG-1 treatment (p<0.01). B, Increased type IV collagen synthesis in STZ-treated animals (p<0.001) was significantly downregulated by insulin and NRG-1 (p<0.01).

FIG. 6: Presence of NRG-1 specific receptors and inhibition of Ang II-stimulated collagen synthesis by NRG-1 in renal glomerular mesangial cells. A, Presence of ErbB2, ErbB3 and ErbB4 in mesangial cells. B, NRG-1 prevented collagen 1a1 synthesis by Ang II over a time period of 48 hours in mesangial cells (p<0.001).

FIG. 7: Amino acid sequences of a neuregulin fragment (SEQ ID NO: 1) and human neuregulin-1 (SEQ ID NO: 2).

FIG. 8: Plasma creatinine in control and CIN-groups, untreated or treated with NRG-1. There was a significant increase in the plasma creatinine of the CIN treated animals, which was completely prevented by NRG-1 treatment. NRG-1 has no effect on the plasma creatinine concentrations in healthy control animals. * P<0.05, ** p<0.01, *** p<0.001.

FIG. 9: Glomerular filtration rate (GFR) of mice which have been water deprived for 24 hours to activate the renal angiotensin system (control) untreated or treated with NRG-1. Results show that NRG-1 induces a significant increase GFR. * P<0.05.

FIG. 10: Weight of the obstructed kidney (UUO) in comparison with their contralateral significant increase in weight in comparison with the control kidneys. NRG-1 treatment did not affect kidney weight. * P<0.05, ** p<0.01, *** p<0.001.

FIG. 11: Plasma creatinine concentrations in sham operated animals in comparison with UUO-animals, untreated or treated with NRG-1.

FIG. 12: Relative mRNA level of inflammation and fibrotic markers in control and obstructed kidneys. Obstruction of the left kidney for 7 days resulted in a significant upregulation of TGF-beta, ICAM-1 and VCAM-1, markers of inflammation, and upregulation of procollagen 1a1, 3a1 and fibronectin, which indicate development of renal fibrosis. NRG-1 treatment protects the obstructed kidney against mRNA synthesis of TGF-beta, ICAM-1 and procollagen 3a1. * P<0.05, ** p<0.01, *** p<0.001

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); Innis et al., PCR Protocols: A Guide to Methods and Applications, Academic Press: San Diego, 1990. General principles of microbiology are set forth, for example, in Davis, B. D. et al., Microbiology, 3rd edition, Harper & Row, publishers, Philadelphia, Pa. (1980).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

In a first aspect, the present invention relates to a neuregulin (NRG) protein, or a functional fragment or homologue thereof for use in a method of treating, preventing and/or delaying nephropathy in a mammal.

As used herein, the terms “treating” or “treatment” refer to therapeutic treatment. The terms “treatment”, “treating”, and the like, as used herein also include amelioration or elimination of a developed disease or condition once it has been established or alleviation of the characteristic symptoms of such disease or condition. The terms “preventing” or “prevention” refer to prophylactic measures, wherein the object is to prevent or slow down (lessen) an undesired physiological change or disorder. As used herein these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of the compound or composition of the invention to a patient that is not at the time of administration afflicted with the disease or condition. “Preventing” also encompasses preventing the recurrence or relapse-prevention of a disease or condition or of symptoms associated therewith, for instance after a period of improvement. The terms “delaying” or “delay” may equally refer to postponing the onset of the disease or symptoms, as well as slowing down the progression of the disease or the symptoms. A subject such as human or animal “in need of treatment” includes ones that would benefit from treatment of a given condition.

As detailed herein the neuregulin proteins, functional fragments or homologues thereof are useful for treating, preventing, and/or delaying nephropathy in a mammal, and/or for treating, preventing, and/or delaying the onset or progression of nephropathy in a mammal. It is envisaged that the neuregulin protein, functional fragment or homologue thereof is useful for the treatment and prevention of both nephropathy as a primary symptom as well as for the treatment and prevention of secondary nephropathy, optionally in combination with other medication which does not directly treat or prevent nephropathy.

As used herein, the term “nephropathy” refers to kidney disease (also known as renal or kidney dysfunction, which may also be interchangeably known as renal or kidney failure or insufficiency), generally encompasses states, diseases and conditions in which the functioning of renal tissue is inadequate, particularly wherein kidney excretory function is compromised. Signs and symptoms of renal dysfunction may include without limitation any one or more of increased levels of urea and/or nitrogen in the blood; lower than normal creatinine clearance and higher than normal creatinine levels in blood; lower than normal free water clearance; volume overload and swelling; abnormal acid levels; higher than normal levels of potassium, calcium and/or phosphate in blood; changes in urination (e.g., volume, osmolarity); microalbuminuria or macroalbuminuria; altered activity of kidney enzymes such as gamma glutamyl synthetase; fatigue; skin rash or itching; nausea; dyspnea; reduced kidney size; haematuria and anaemia. As used herein, nephropathy is deemed as comprising major classes denoted as acute renal or kidney failure (acute renal or kidney disease or injury, e.g., acute kidney injury or “AKI”) or chronic renal or kidney failure (chronic renal or kidney disease). Whereas progression is typically fast (e.g., days to weeks) in acute renal failure, renal failure may be traditionally regarded as chronic if it persists for at least 3 months and its progression may take in the range of years. As used herein, nephropathy may refer to nephrosis, i.e. non-inflammatory nephropathy, or nephritis, i.e. inflammatory nephropathy. The diagnosis of both acute and chronic kidney disease is known in the art, such that a subject having nephropathy can be readily identified. By means of further guidance, nephropathy in general involves among others a reduction in glomerular filtration rate (GFR). Acute and chronic nephropathy may be defined as follows.

Acute renal dysfunction or failure may be staged (classified, graded) into 5 distinct stages using the “RIFLE” (Risk, Injury, Failure, Loss, end-stage renal disease) staging system as set out here below (based on Lameire et al. 2005, Lancet 365: 417-430):

| | GFR (based on serum | |
|---|---|---|
| Stage | creatinine) criteria | Urine output criteria |
| “Risk” | Serum creatinine increased 1.5 times | <0.5 mL/kg/h for 6 h |
| “Injury” | Serum creatinine increased 2.0 times | <0.5 mL/kg/h for 12 h |
| “Failure” | Serum creatinine increased 3.0 times, | <0.3 mL/kg/h for 24 h |
| | or creatinine >355 mM/L when there | or anuria for 12 h |
| | was an acute rise of >44 mM/L | |
| “Loss” | Persistent acute renal failure >4 | — |
| | weeks | |
| “End- | End-stage renal disease >3 months | — |
| stage” | | |

Chronic renal dysfunction or failure may be staged (classified, graded) based on GFR as set out here below (based on Levey et al. 2005, Kidney Int 67: 2089-2100):

Stage 1: GFR=90 mL/min (normal or elevated GFR)

Stage 2: GFR=60-89 mL/min (mild GFR reduction)

Stage 3: GFR=30-59 mL/min (moderate GFR reduction)

Stage 4: GFR=15-29 mL/min (severe GFR reduction)

Stage 5: GFR<15 mL/min (renal failure)

The origin of the nephropathy to be treated or prevented as described herein is not critical. In particular embodiments, the nephropathy is selected from the group comprising or consisting of diabetic nephropathy (nephrangiosclerosis), nephropathy caused by hypertension, nephropathy caused by vasculitis, lupus nephritis, nephropathy caused by glomerulonephritis, nephropathy caused by tubuloinsterstitial diseases, obstructive nephropathy, contrast induced nephropathy, glomerulonephritis, acute tubular necrosis (ATN), and acute interstitial nephritis (AIN). This list of conditions all fall in the general category of nephropathy. Nephropathy may however in some cases be secondary to a primary condition which a subject is afflicted with. For instance, diabetics often develop secondary nephropathy, which is then termed “diabetic nephropathy”. While the above listed conditions may differ in some clinical aspects, common to all is the inadequate functioning of the kidneys. In a preferred embodiment, the nephropathy is diabetic nephropathy.

Associated with inadequate kidney functioning may for instance be albuminuria, glomerulosclerosis, or renal fibrosis. Accordingly, in particular embodiments, the nephropathy as referred to herein is characterized by one or more of albuminuria, glomerulosclerosis, and/or renal fibrosis. In particular embodiments, the nephropathy is characterized by albuminuria. In further embodiments, the nephropathy is characterized by glomerulosclerosis. In further embodiments, the nephropathy is characterized by renal fibrosis. In particular embodiments, the nephropathy is characterized by albuminuria and glomerulosclerosis. In particular embodiments, the nephropathy is characterized by albuminuria and renal fibrosis. In particular embodiments, the nephropathy is characterized by glomerulosclerosis and renal fibrosis. In further embodiments, the nephropathy is characterized by albuminuria, glomerulosclerosis, and renal fibrosis.

It has been established herein that administration of a neuregulin protein is effective in the reduction, prevention or treatment of mammals suffering from either mild or severe for the treatment of a mammal suffering from mild or severe GFR, which comprise administering a neuregulin protein to said mammal.

The effect of the treatment methods envisaged as detailed herein is reduced or prevention of nephropathy. This effect can be assessed in different ways. Examples of criteria and/or endpoints which have been used in the assessment of kidney disease progression include but are not limited to Progression to kidney failure (GFR<15 ml/min per 1.73 m2 or the initiation of dialysis or transplantation), changes in proteinurea and/or albiminurea, levels of GFR or S_{cr} (serum creatinine (mg/dL)). Accordingly, in particular embodiments, methods and compositions are provided whereby administration of a neuregulin protein to a mammal ensures a decrease or prevents an increase in kidney disease progression as determined by one or more of these criteria in said patient.

The present inventors have found that administration of a neuregulin protein to a mammal prevents or reduces renal fibrosis as can be established by a reduction or prevention of the upregulation of renal fibrosis markers FSP-1 and Collagen type IV RNA indicative of collagen synthesis. Urinary FSP-1 has also been found to be indicative of kidney function. Accordingly, in particular embodiments, methods and compositions are provided whereby administration of a neuregulin protein to a mammal suppresses collagen synthesis, preferably collagen IV synthesis, and/or FSP-1 synthesis.

As used herein, “synthesis” refers to protein expression. Suppression of protein synthesis may relate to suppression of transcription of the protein encoding gene and/or suppression of translation of the protein encoding mRNA, both of which can be assayed by routine techniques, such as respectively Western blot or Q-PCR. In a preferred embodiment, suppression of collagen synthesis, preferably collagen type IV synthesis, and/or FSP-1 synthesis relates to a suppression or reduction of transcription of the respective genes. Suppression of one or both of these genes preferably occurs in renal cells, more preferably renal glomerular cells or renal mesangial cells, even more preferably renal glomerular mesangial cells. Accordingly, in particular embodiments of the methods envisaged herein, the neuregulin protein suppresses collagen, preferably collagen IV, and/or FSP-1 synthesis in renal cells. More particularly renal glomerular cells. In particular embodiments of the methods envisaged herein, the neuregulin protein suppresses collagen, preferably collagen IV, and/or FSP-1 synthesis in renal mesangial cells.

In particular embodiments, the application envisages the prevention of nephropathy, more particularly in subjects susceptible to developing nephropathy. Examples of subjects susceptible to nephropathy include subjects which come into contact with one or more factors known to induce acute kidney injury such as the following: (1) A sudden, serious drop in blood flow to the kidneys Heavy blood loss, an injury, or a bad infection called sepsis can reduce blood flow to the kidneys. Not enough fluid in the body (dehydration) also can harm the kidneys. Also pregnancy complications such as eclampsia or pre-eclampsia may lead to acute kidney failure. (2) Damage from some medicines, poisons, or infections, more particularly subjects who have serious, long-term health problems. Examples of medication that can induce nephropathy include antibiotics, such as gentamicin and streptomycin; pain medicines, such as naproxen and ibuprofen; some blood pressure medicines, such as ACE inhibitors; or the dyes used in some X-ray tests. (3) A sudden blockage that stops urine from flowing out of the kidneys. Kidney stones, a tumor, an injury, or an enlarged prostate gland can cause a blockage. Additionally or alternatively, subjects susceptible to nephropathy include subjects which have diabetes or high blood pressure. Additionally or alternatively, subjects susceptible to nephropathy include subjects which come suffer from a disease or come into contact with one or more factors known to induce chronic kidney injury such as (1) Immune system conditions such as lupus and chronic viral illnesses such as HIV/AIDS, hepatitis B, and hepatitis C; (2) Urinary tract infections within the kidneys themselves, called pyelonephritis, can lead to scarring as the infection heals. Multiple episodes can lead to kidney damage; (3) Inflammation in the tiny filters (glomeruli) within the kidneys; this can happen after strep infection and other conditions of unknown cause. (4) Polycystic kidney disease, in which fluid-filled cysts form in the kidneys over time. This is the most common form of inherited kidney disease. (5) Congenital defects, present at birth, are often the result of a urinary tract obstruction or malformation that affects the kidneys. One of the most common involves a valve-like mechanism between the bladder and urethra. These defects, sometimes found while a baby is still in the womb, can often be surgically repaired by a urologist. (6) Drugs and toxins, including long-term exposure to some medications and chemicals; overuse of NSAIDs (nonsteroidal anti-inflammatory drugs), such as ibuprofen and naproxen; and use of intravenous “street” drugs. It will be understood to the skilled person that in patients suffering from nephropathy due to one or more of these factors are also envisaged to be treated by the methods of treatment described herein.

Without being bound by theory, it is believed that the neuregulin protein activates various signaling pathways, such as the Akt signaltransduction pathway and the Erk signaltransduction pathway so as to ensure its effect on nephropathy as observed herein. Accordingly, in particular embodiments, the invention relates to a neuregulin protein for use in the reduction, prevention or treatment of nephropathy, wherein said neuregulin protein activates the Akt and/or Erk signaling pathways. Methods for identifying activation of signal transduction pathways are well known in the art. The Akt and Erk signaling pathways are also well known in the art. Accordingly, the skilled person is amply capable of evaluating the activation of either one of these pathways. By means of further guidance, activation of these pathways may for instance be determined by measurement of phosphorylated Akt, respectively Erk. In this context, phosphorylation (or increased phosphorylation) of Akt or Erk indicates activation (or increased activation) of respectively the Akt and Erk pathways. Activation of one or both of these pathways preferably occurs in renal cells, more preferably renal glomerular cells or renal mesangial cells, even more preferably renal glomerular mesangial cells.

As used herein, the term “neuregulin protein” refers to a protein of the neuregulin family. Neuregulins or neuroregulins are a family of four structurally related proteins that are part of the EGF family of proteins. The neuregulin family includes: (1) neuregulin-1 (NRG-1), with isoforms stemming from alternative splicing: type I NRG-1; alternative names: Heregulin, NEU differentiation factor (NDF), or acetylcholine receptor inducing activity (ARIA); type II NRG-1; alternative name: Glial Growth Factor-2 (GGF2); type III NRG-1; alternative name: Sensory and motor neuron-derived factor (SMDF); type IV NRG-1; type V NRG-1; type VI NRG-1; (2) Neuregulin-2 (NRG-2); (3) Neuregulin-3 (NRG-3); (4) Neuregulin-4 (NRG-4).

In certain embodiments, the neuregulin protein as referred to herein may be either one or a mixture of two or more of the above recited family members. It is to be understood that the neuregulin protein as referred to herein is preferably the mature neuregulin protein (i.e. the cleaved pro-neuregulin protein, which contains the EGF-like domain), which may or may not contain a signal peptide, but preferably does not contain a signal peptide. The neuregulin protein as referred to herein may be a naturally occurring neuregulin protein, for instance which is isolated from a specific host. Alternatively, the neuregulin protein as referred to herein may be recombinantly produced (e.g. in E. coli, yeast, CHO cell lines, or other hosts). In a preferred embodiment, the neuregulin protein as used herein is NRG-1. In a more preferred embodiment, the neuregulin protein as used herein is type I NRG-1 (heregulin). In an even more preferred embodiment, the neuregulin protein as used herein is the beta isoform of NRG-1, preferably NRG-1 type I, i.e. NRG-1 type I β. In a further preferred embodiment, the neuregulin protein as used herein is the beta1 isoform of NRG-1, preferably NRG-1 type I, i.e., NRG-1 type I β1.

In particular embodiments, the neuregulin protein as referred to herein is a human neuregulin protein. In a preferred embodiment, the neuregulin protein as used herein is human NRG-1. In a more preferred embodiment, the neuregulin protein as used herein is type I human NRG-1 (heregulin). In an even more preferred embodiment, the neuregulin protein as used herein is the beta isoform of human NRG-1, preferably human NRG-1 type I, i.e. human NRG-1 type I β. In a further preferred embodiment, the neuregulin protein as used herein is the beta1 isoform of human NRG-1, preferably human NRG-1 type I, i.e. human NRG-1 type I β1.

The application also envisages the use of a homologue, an orthologue, or a functional fragment or variant of a neuregulin protein, such as of a human neuregulin protein. The terms “orthologue”, “homologue”, “functional variant”, and “functional fragment” are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. A “functional variant” or “functional fragment” of a protein as used herein refers to a variant or fragment of such protein which retains or mimics at least partially the activity of that protein. Functional variants or fragments may include mutants (which may be insertion, deletion, or replacement mutants), including polymorphs, etc. Functional variants or fragments may be naturally occurring or may be man-made. In the context of the present invention, a functional fragment, refers to a fragment of a neuregulin protein which can bind to and activate a cognate ErbB receptor. Similarly, a functional variant or a homologue, refers to a molecule which can bind to and activate a cognate ErbB receptor.

In particular embodiments, the homologue, orthologue, functional variant, or functional fragment of the neuregulin protein as referred to herein has a sequence identity of at least 80%, more preferably at least 85%, even more preferably at least 90%, such as for instance at least 95% with one or more of the human neuregulin proteins. It is to be understood that when referring to sequence alignments, the sequence identity is to be determined based on the shortest sequence to be aligned. For instance, sequence alignment of a neuregulin fragment which is shorter than the neuregulin full length protein is to be determined based on the length of the fragment. In a preferred embodiment, the homologue, orthologue, functional variant, or functional fragment of the neuregulin protein as referred to herein has a sequence identity of at least 80%, more preferably at least 85%, even more preferably at least 90%, such as for instance at least 95% with human NRG-1. In a more preferred embodiment, the homologue, orthologue, functional variant, or functional fragment of the neuregulin protein as referred to herein has a sequence identity of at least 80%, more preferably at least 85%, even more preferably at least 90%, such as for instance at least 95% with type I human NRG-1 (heregulin). In an even more preferred embodiment, the homologue, orthologue, functional variant, or functional fragment of the neuregulin protein as referred to herein has a sequence identity of at least 80%, more preferably at least 85%, even more preferably at least 90%, such as for instance at least 95% with or is identical to the beta isoform of human NRG-1, preferably human NRG-1 type I, i.e. human NRG-1 type I β, which corresponds to the N-terminal fragment of NRG-1. In a further preferred embodiment, the homologue, orthologue, functional variant, or functional fragment of the neuregulin protein as referred to herein has a sequence identity of at least 80%, more preferably at least 85%, even more preferably at least 90%, such as for instance at least 95% with the beta1 isoform of human NRG-1, preferably human NRG-1 type I, i.e. human NRG-1 type I β1. In a further preferred embodiment, the homologue, orthologue, functional variant, or functional fragment of the neuregulin protein as referred to herein has a sequence identity of at least 80%, more preferably at least 85%, even more preferably at least 90%, such as for instance at least 95%, more particularly is 100% identical to SEQ ID NO:2. In particular embodiments, the neuregulin protein, functional fragment, functional variant, orthologue, or homologue as referred to herein, such as a human neuregulin protein, functional fragment, functional variant, orthologue, or homologue comprises, consists essentially of, or consists of an EGF-like domain. EGF-like domains are well known in the art and can be easily identified by routine techniques involving sequence alignments. A protein BLAST analysis also outputs conserved domains, such that the presence of an EGF-like domain can be readily evaluated. The EGF-like domains of all neuregulins have for instance also been annotated in protein and nucleic acid databases, which can for instance be accessed at the ncbi website. The skilled person is therefore capable to easily determine if the neuregulin protein, functional fragment, functional variant, orthologue, or homologue as referred to herein comprises an EGF-like domain.

In particular embodiments, the EGF-like domain containing homologue, orthologue, functional variant, or functional fragment of the neuregulin protein as referred to herein has a sequence identity of at least 80%, more preferably at least 85%, even more preferably at least 90%, such as for instance at least 95% with human neuregulin. In a preferred embodiment, the EGF-like domain containing homologue, orthologue, functional variant, or functional fragment of the neuregulin protein as referred to herein has a sequence identity of at least 80%, more preferably at least 85%, even more preferably at least 90%, such as for instance at least 95% with human NRG-1. In a more preferred embodiment, the EGF-like domain containing homologue, orthologue, functional variant, or functional fragment of the neuregulin protein as referred to herein has a sequence identity of at least 80%, more preferably at least 85%, even more preferably at least 90%, such as for instance at least 95% with type I human NRG-1 (heregulin). In an even more preferred embodiment, the EGF-like domain containing homologue, orthologue, functional variant, or functional fragment of the neuregulin protein as referred to herein has a sequence identity of at least 80%, more preferably at least 85%, even more preferably at least 90%, such as for instance at least 95% with the beta isoform of human NRG-1, preferably human NRG-1 type I, i.e. human NRG-1 type I β. Examples of functional variants of a neuregulin protein are provided in US2014031284, WO03/099300, US537060 and U.S. Pat. No. 6,136,558.

In a further preferred embodiment, the EGF-like domain containing functional fragment of the neuregulin protein as referred to herein has a sequence identity of at least 80%, more preferably at least 85%, even more preferably at least 90%, such as for instance at least 95% with the beta1 isoform of human NRG-1, preferably human NRG-1 type I, i.e. human NRG-1 type I β1. In a particular embodiment, the functional fragment of the neuregulin protein as referred to herein corresponds to the sequence of the EGF domain of human neuregulin-1 or Heregulin-β1. In a preferred embodiment, the functional fragment of the neuregulin protein as referred to herein has a sequence identity of at least 80%, more preferably at least 85%, even more preferably at least 90%, such as for instance at least 95%, more particularly 100% sequence identity with the sequence of SEQ ID NO: 1.

In particular embodiments, the homologue of the neuregulin protein is a protein or compound capable of binding to and activating the ErbB4 receptor. Examples of proteins and molecules which can be identified based on their ability to bind and activate the ErbB4 receptor are activating antibodies or small molecules. In particular embodiments, these molecules specifically activate the ErbB4 receptor.

The neuregulin protein as taught herein may be used in monomeric form or in multimeric or multivalent form, preferably in dimeric or bivalent form. Dimers of a neuregulin protein are not known to be naturally occurring and, as a result, are referred to herein as being synthetic or engineered. In certain embodiments, the neuregulin protein is used in dimeric form. Neuregulin multimers or dimers as described herein comprise a neuregulin protein in monomeric form and one or more of the same or another ErbB2, ErbB3 or ErbB4 ligand. The monomers of the neuregulin dimer may be identical (i.e. neuregulin homodimer) or different (i.e. neuregulin heterodimer). Accordingly, contemplated herein are the following non-limiting examples of neuregulin dimers: NRG2b-NRG2b, NRG2b-NRG2a, NRG2b-NRG1B3, NRG2b-NRG1α, NRG2b-NRG1B, NRG2b-NRG2, NRG2b-NRG3, NRG2b-NRG4, NRG2a-NRG2a, NRG2a-NRG1B3, NRG2a-NRG1α, NRG2a-NRG1B, NRG2a-NRG2, NRG2a-NRG3, NRG2a-NRG4, NRG1B3-NRG1B3, NRG1B3-NRG1α, NRG1B3-NRG1B, NRG1B3-NRG2, NRG1B3-NRG3, NRG1B3-NRG4, NRG1a-NRG1α, NRG1a-NRG1B, NRG1a-NRG2, NRG1a-NRG3, NRG1a-NRG4, NRG1B-NRG1B, NRG1B-NRG2, NRG1B-NRG3, NRG1B-NRG4, NRG2-NRG2, NRG2-NRG3, NRG2-NRG4, NRG3-NRG3, NRG3-NRG4, and NRG4-NRG4. The neuregulin monomers are typically linked with a linker in the neuregulin dimers described herein. The linker may comprise a coiled coil, a peptide spacer, a water soluble flexible polymer (such as e.g. polyethylene oxide, dextran, polyacrylic acid and polyacrylamide), or a combination thereof. The neuregulin dimers can be produced with e.g. the methods described in paragraphs 104 to 107 of US application US 2013/0196911, which is specifically incorporated by reference herein, or methods otherwise described in the art. Methods for producing ligand dimers such as neuregulin dimers are known in the art and described in e.g. PCT application WO2010033249, which is specifically incorporated by reference herein.

Methods for comparing sequences and determining sequence identity are well known in the art. By means of example, percentage of sequence identity refers to a percentage of identical nucleic acids or amino acids between two sequences after alignment of these sequences. Alignments and percentages of identity can be performed and calculated with various different programs and algorithms known in the art. Preferred alignment algorithms include BLAST (Altschul, 1990; available for instance at the NCBI website) and Clustal (reviewed in Chenna, 2003; available for instance at the EBI website). Preferably, BLAST is used to calculate the percentage of identity between two sequences, such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

In particular embodiments, the neuregulin protein, or the homologue, orthologue, functional variant, or functional fragment of the neuregulin protein as referred to herein comprises, consists essentially of, or consists of a polypeptide having a sequence as set forth in SEQ ID NO: 1 or SEQ ID NO: 2, or has a sequence identity of at least 80%, more preferably at least 85%, even more preferably at least 90%, such as for instance at least 95% with a polypeptide having a sequence as set forth in SEQ ID NO: 1 or SEQ ID NO: 2.

A neuregulin protein as described herein, the homologue, orthologue, functional variant, or functional fragment of the neuregulin protein as referred to herein optionally together with a pharmaceutically acceptable carrier may be administered by any suitable mode of application, e.g. i.d., i.v., i.p., i.m., intranasally, orally, subcutaneously, etc. and in any suitable delivery device (O'Hagan et al., Nature Reviews, Drug Discovery 2 (9), (2003), 727-735). The proteins of the present invention are preferably formulated for intravenous, subcutaneous, intradermal or intramuscular administration (see e.g. “Handbook of Pharmaceutical Manufacturing Formulations”, Sar-faraz Niazi, CRC Press Inc, 2004). Accordingly, the present invention also relates to a pharmaceutical composition comprising the a neuregulin protein as described herein, the homologue, orthologue, functional variant, or functional fragment of the neuregulin protein as referred to herein, optionally together with a pharmaceutically acceptable carrier, fur use in treating, preventing, and/or delaying nephropathy in a mammal. As used herein, “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutically active substances is well known in the art. Such materials should be non-toxic and should not interfere with the activity of the neuregulins.

In an aspect, the invention also relates to a pharmaceutical composition comprising the neuregulin protein, homologue, orthologue, functional variant, or functional fragment thereof, as defined herein elsewhere, in an effective amount for use in treating, preventing and/or delaying nephropathy in a mammal.

As used herein, the term “effective amount” refers to the amount or dose of the protein (or nucleic acid) or a composition, such as a pharmaceutical composition which attains a therapeutic or prophylactic effect in a subject to which it is administered. An effective amount is an amount which can elicit a biological or medicinal response in a tissue, system, animal or human to which the protein, nucleic acid, or composition is administered, and in particular can prevent or alleviate one or more of the local or systemic symptoms or features of a disease or condition being treated.

In an embodiment, the neuregulin protein, homologue, orthologue, functional variant, or functional fragment thereof, as defined herein elsewhere, is to be administered in a concentration ranging from 0.01 to 100 μg/kg, i.e from 0.01 to 100 μg/kg body weight of the subject it is to be administered to, preferably from 0.05 to 50 μg/kg, more preferably from 0.1 to 10 μg/kg. In another embodiment, the neuregulin protein, homologue, orthologue, functional variant, or functional fragment thereof, as defined herein elsewhere, is to be administered in a concentration ranging from 0.01 to 100 μg/kg/day, i.e from 0.01 to 100 μg/kg body weight of the subject it is to be administered to per day, preferably from 0.05 to 50 μg/kg/day, more preferably from 0.1 to 10 μg/kg/day. In another embodiment, the neuregulin protein, homologue, orthologue, functional variant, or functional fragment thereof, as defined herein elsewhere, is to be administered in a concentration ranging from 0.01 to 100 μg/kg/week, i.e from 0.01 to 100 μg/kg body weight of the subject it is to be administered to per week, preferably from 0.05 to 50 μg/kg/week, more preferably from 0.1 to 10 μg/kg/week.

In an embodiment, the neuregulin protein, homologue, orthologue, functional variant, or functional fragment thereof, as defined herein elsewhere, is to be administered in a concentration ranging from 10 to 1000 pmol/kg, i.e from 10 to 1000 pmol/kg body weight of the subject it is to be administered to, preferably 30 to 500 pmol/kg, more preferably from 50 to 100 pmol/kg. In another embodiment, the neuregulin protein, homologue, orthologue, functional variant, or functional fragment thereof, as defined herein elsewhere, is to be administered in a concentration ranging from 10 to 1000 pmol/kg/day, i.e from 10 to 1000 pmol/kg body weight of the subject it is to be administered to per day, preferably 30 to 500 pmol/kg/day, more preferably from 50 to 100 pmol/kg/day. In another embodiment, the neuregulin protein, homologue, orthologue, functional variant, or functional fragment thereof, as defined herein elsewhere, is to be administered in a weight of the subject it is to be administered to per week, preferably 30 to 500 pmol/kg/week, more preferably from 50 to 100 pmol/kg/week.

It will be understood by the skilled person that the duration of the treatment may vary, possibly depending on the desired outcome, for instance improvement of one or more symptoms, complete cure, etc. For instance, the neuregulin protein, such as a pharmaceutical composition comprising a neuregulin protein, may be administered only once. Alternatively, the neuregulin protein may be administered on a daily basis for a specified duration, such as for instance during or at least during 2, 3, 4, 5, 6, 7, or more days, which may or may not be consecutive days. The neuregulin protein may also be administered multiple times per day, such as at least 2, 3, 4, 5, 6, 7 or more times per day. The neuregulin protein may for instance also be administered multiple times per week, such as for instance at least 2, 3, 4, or more times per week. The neuregulin protein may for instance also be administered weekly, every 2, 3, 4 or more weeks. The neuregulin protein may for instance also be administered monthly, every 2, 3, 4 or more months.

It will be further understood by the skilled person that the mode of administration of the neuregulin protein may vary. For instance, the neuregulin protein, such as a pharmaceutical composition comprising a neuregulin protein, may be administered in bolus, or may alternatively be administered during a prolonged time frame. For instance, the neuregulin protein may be administered, e.g. as a drip, over a period of several minutes or hours, such as for instance during 5, 6, 7, 8, 9, 10, 11, 12, 13, 14, 15, 16, 17, 18, 19, 20, or more minutes, such as for instance during 10, 20, 30, 40, 50, 60, or more minutes, such as for instance during, 0.5, 1, 1.5, 2, 2.5, 3, 3.5, 4, or more hours, such as for instance during 4, 6, 8, 10, 12, 14, 16, 18, 20, 22, 24, or more hours.

In particular embodiments, patients suffering from nephropathy such as diabetic nephropathy are treated by administering the neuregulin protein by IV dosing of 0.3-1.0 ug/kg/day, 5 days, 10 hour drip. In particular embodiments, the treatment involves administration of a first lower dosage (between of 0.3-0.7 ug/kg/day), followed by administration of a slightly higher dosage (between 0.5-1.0 ug/kg/day). In further particular embodiments, for patients suffering from diabetic nephropathy characterized by severe reduction in GFR (15-30 ml/min), the neuregulin protein is administered to patients in need thereof by IV dosing, 0.6 ug/kg/day neuregulin-1 (5 days, 10 hour drip) followed by 0.8 ug/kg (weekly, 10 min drip, 20 weeks). In particular embodiments, for patients suffering from diabetic nephropathy characterized by normal GFR or mildly reduced GFR (GFR>60 ml/min) and albuminuria, the neuregulin protein is administered to patients in need thereof by IV dosing, 0.8 ug/kg/day neuregulin-1 (weekly, 10 min drip, 20 weeks).

In another aspect, the invention relates to a nucleic acid comprising a nucleic acid sequence encoding a neuregulin protein as described herein, the homologue, orthologue, functional variant, or functional fragment of the neuregulin protein as referred to herein for use in treating, preventing, and/or delaying nephropathy in a mammal. Preferably, said nucleic acid is a eukaryotic expression vector which comprises a nucleic acid sequence encoding a neuregulin protein as described herein, the homologue, orthologue, functional variant, or functional fragment of the neuregulin protein as referred to herein. Such vectors are well known in the art, and may include regulatory elements or tissue specific promoters such that expression of the encoded sequence can be modulated, such as to result in tissue specific expression, but also inducible expression, or combinations thereof.

Additionally the invention relates to methods for treating, preventing and/or delaying nephropathy, as described herein above comprising administering to a subject in need thereof a neuregulin protein, homologue, orthologue, functional variant, or functional fragment thereof, as defined herein elsewhere, or a nucleic acid encoding such protein, as defined herein elsewhere. In particular embodiments, the methods thereby treat, prevent and/or delay nephropathy. In further embodiments, the methods involve, determining a nephropathy or a susceptibility to nephropathy in a subject and thereafter administering said neuregulin protein, homologue, orthologue, functional variant, or functional fragment thereof, as defined herein elsewhere. The types of nephropathy envisaged in the methods provided herein are detailed elsewhere.

Additionally, the invention relates to the use of a neuregulin protein, homologue, orthologue, functional variant, or functional fragment thereof, as defined herein elsewhere, or a nucleic acid encoding such protein, as defined herein elsewhere for the preparation of a medicament for treating, preventing and/or delaying nephropathy, as defined herein elsewhere.

The aspects and embodiments of the invention are further supported by the following non-limiting examples.

### EXAMPLES

### Example 1

### Prevention of Nephropathy Associated with Type I Diabetes

The ability of neuregulin to prevent nephropathy, in particular diabetic nephropathy was determined in an animal model for Type 1 Diabetes Mellitus.

Animals

Laboratory bred male Apolipoprotein (Apo)E deficient mice were initially obtained from Jax Laboratories. Mice were maintained under standard laboratory conditions, 12 hour light-dark cycles with access to normal chow and drinking water at libitum. All experiments performed are approved by the ethical committee of animals (ECD) of the university of Antwerp conform to the Guide for the Care and Use of Laboratory Animals published by the US National Institutes of Health (NIH Publication No. 85-23, revised 1996).

Induction of Type 1 Diabetes Mellitus

16 week old ApoE mice (n=62) received intraperitoneal injections of streptozotocin (STZ, 60 mg/kg, Sigma Aldrich) in citrate buffer (0.05M) for 5 consecutive days. Before STZ injection, mice were fasted for 6 hours. 2 weeks after the initial injection, mice developed hyperglycemia. In the case that blood glucose levels did not exceed values higher than 300 mg/dl, mice were excluded from the study.

Kidney Function Markers

24 hour urine of each animal was collected in metabolic cages at week 14, the day before sacrifice. Upon sacrifice, blood samples were obtained by cardiac puncture under terminal anaesthesia and collected in the presence of heparin. After centrifugation of the blood samples, plasma was isolated. Urinary microalbuminuria (Bethyl Laboratories) and Neutrophil gelatinase-associated lipocalin (NGAL, Abcam) ELISA's were performed according to the manufacturers protocol. Both urinary and serum creatinine were measured to determine creatinine clearance. Urinary creatinine was analyzed via the colorimetric Jaffé method, while plasma creatinine was defined via autoanalyzer (Siemens Vista 1500).

Histological Assessment of Renal Injury and Presence of ErbB Receptors

Left kidneys were weighted and fixed in 4% buffered formalin and embedded in paraffin for histological staining according to standard procedures. Kidney sections (5 μm) were stained with Masson's trichrome in order to determine the degree of glomerulosclerosis. 20 images at a 40× magnification of outer cortical glomerular cross-sections per kidney where captured via light microscopy (Olympus U-TU1X-2, Japan). Unbiased histological quantification was performed by using ImageJ as previously described (7). Glomerular positivity was expressed as the ratio of the percentage of positive staining to the glomerular tuft area.

Presence of the NRG-1 specific receptor ErbB4 within the glomerular tuft area was determined by immunohistological staining with an anti-ErbB4 antibody (ErbB4 (C-18): sc-283, Santa Cruz). To avoid false-positive results with unspecific binding of the secondary antibody, ErbB4-stained kidneys were compared with blank-stained images, consisting of incubation with the secondary antibody only.

Renal Mesangial Cells

Mouse renal glomerular mesangial cells were purchased (P10628, Innoprot) and cultured according to the manufacturers protocol. In brief, cells were thawed upon arrival in a 37° C. water bad and plated in Poly-L Lysine coated flasks in Dulbecco's modified Eagle's medium (DMEM) enriched with 10% Fetal bovine serum (FBS). Medium was changed every 2 to 3 days.

Presence of ErbB receptors in mesangial cells was analyzed by Western Blotting. In order to assess collagen synthesis, mesangial cells were stimulated with angiotensin II (Ang II, 100 nM, Sigma Aldrich) in the presence or absence of rhNRG-1β for 24, 48 and 72 hours after 24 hours on serum-free DMEM. Collagen RNA expression was measured by Real-Time PCR.

Western Blotting

Mesangial cells were collected in lysis buffer consisting of 20 mM Tris, 137 mM NaCl, 10% (vol/vol) glycerol, 1% (vol/vol) Nonidet P-40, and 2 mM ethylenediaminetetraacetic acid and supplemented with protease and phosphatase inhibitors (Complete; Roche and Sigma, respectively). Western Blotting was performed as previously described (8). In brief, cell lysates were heat-denaturated and loaded on 4-12% NuPage gels (Invitrogen). After electrophoresis, proteins were electrotransferred to polyvinylidene fluoride membranes. Membranes were blocked with 5% BSA and incubated with primary antibodies whereafter secondary horseradish peroxidase-conjugated antibody was applied. Antibodies used were ErbB2, ErbB3 and ErbB4 (Santa Cruz, Abcam).

RNA Extraction and Real-Time PCR

Total RNA was isolated from kidneys or mesangial cells. Right kidneys were snap-frozen in liquid nitrogen at sacrifice. Kidney tissue was then homogenized using a Polytron homogenizer (Pt 2100; Kinematica, Littau, Switzerland) and RNA was obtained by the GenElute Mammalian Total RNA Miniprep Kit (Sigma Aldrich). Mesangial cell RNA was extracted via the Absolutely Microprep RNA kit (Agilent). Total RNA was transcribed to cDNA using random hexamers (TaqMan Reverse Transcription Reagents, Applied Biosystems). Using TaqMan real-time PCR (Life Technologies), collagen type IV (Mm01210125_m1, Col4a1) and Fibroblast specific protein-1 (Mm01210125_m1, s100a4) mRNA expression was analyzed in whole kidney tissue. In mesangial cells collagen synthesis was determined by collagen type I (Mm00801666_g1, Col1a1) expression.

Data Analysis and Statistics

Data are expressed as means±SEM. Differences between groups were analyzed by one-way ANOVA with Bonferroni corrections for multiple comparisons. Western blots were subjected to densitometric analysis using ImageJ 1.42 software. Statistical significance was defined as P<0.05. All statistical analyses were done using Graph Pad Prism 5 and IBM SPSS Statistics 22 software.

Results

Animals with blood glucose levels over 300 mg/dL were considered diabetic.

Diabetic mice were randomized into treatment groups with (i) insulin (n=22, 0.005 U/kg·day, Linshin Canada), (ii) rhNRG-16 (n=19, 20 μg/kg·day, i.p., PreProtech), or (iii) no treatment (n=21) for 14 weeks. Recombinant human Heregulin-β1 (HRG1-β1) is a 7.5 kDa polypeptide consisting of only the EGF domain of Heregulin-β1 (65 amino acid residues corresponding to SEQ ID NO:1).

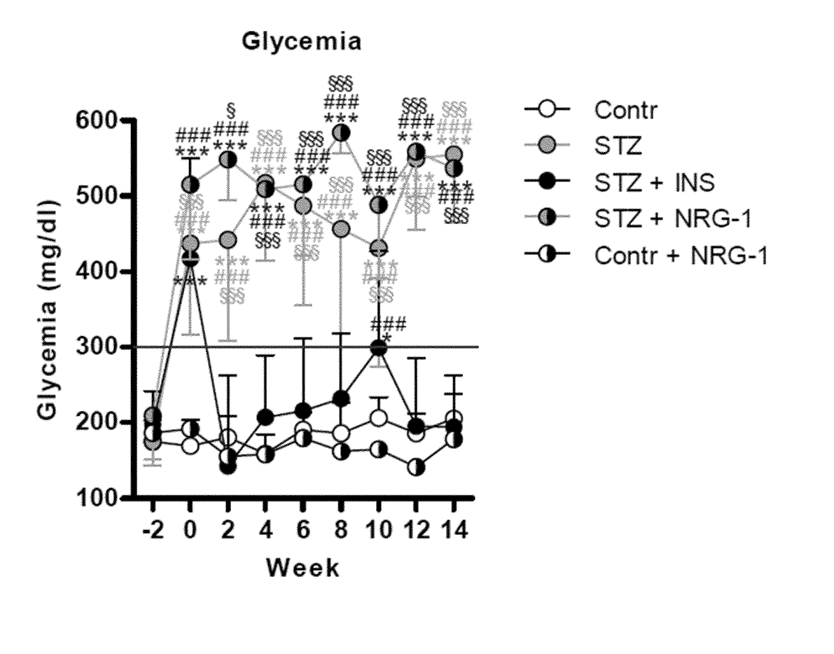

Control non-diabetic ApoE littermates (n=24) received citrate buffer alone and were randomized to either receive (i) or (ii) no treatment over a period of 14 weeks. Animals were monitored for body weight and blood glucose (OneTouch Glucose meter) weekly. Administration of STZ to mice resulted in a significant increase in blood glucose concentrations during the whole experiment in comparison with their non-diabetic littermates as shown in FIG. 1. Insulin treatment significantly reduced hyperglycemia towards control levels, while NRG-1 had no effect on blood glucose, neither in control, nor diabetic animals.

Urinary Markers of Kidney Function

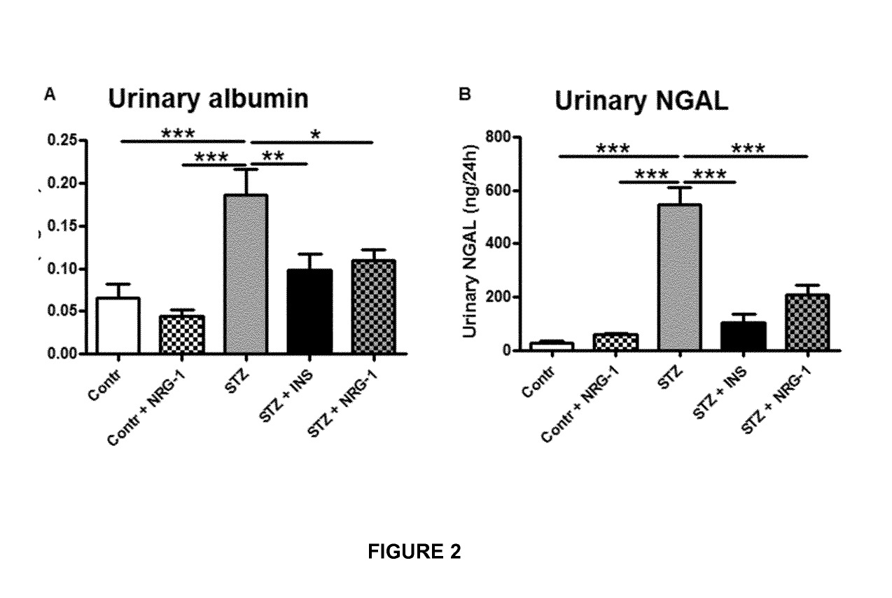

Microalbuminuria was present after 14 weeks in the untreated diabetic animals (0.0925±0.0187 vs 0.196±0.0323 μg/24 h, p<0.05), implicating damage of the glomerular basement membrane. Both insulin and NRG-1 prevented the development of microalbuminuria (0.196±0.0323 vs 0.0985±0.0191 μg/24 h, p<0.01 and 0.196±0.0323 vs 0.109±0.0129 μg/24 h, p<0.05, respectively) (FIG. 2A). A similar trend was observed when urinary NGAL concentrations were determined (FIG. 2B). NGAL is an early marker for kidney dysfunction, as it is synthesized by renal tubuli in response to kidney injury. Creatinine clearances were not impaired in any of the groups.

Histological Assessment of Renal Injury and Presence of ErbB Receptors

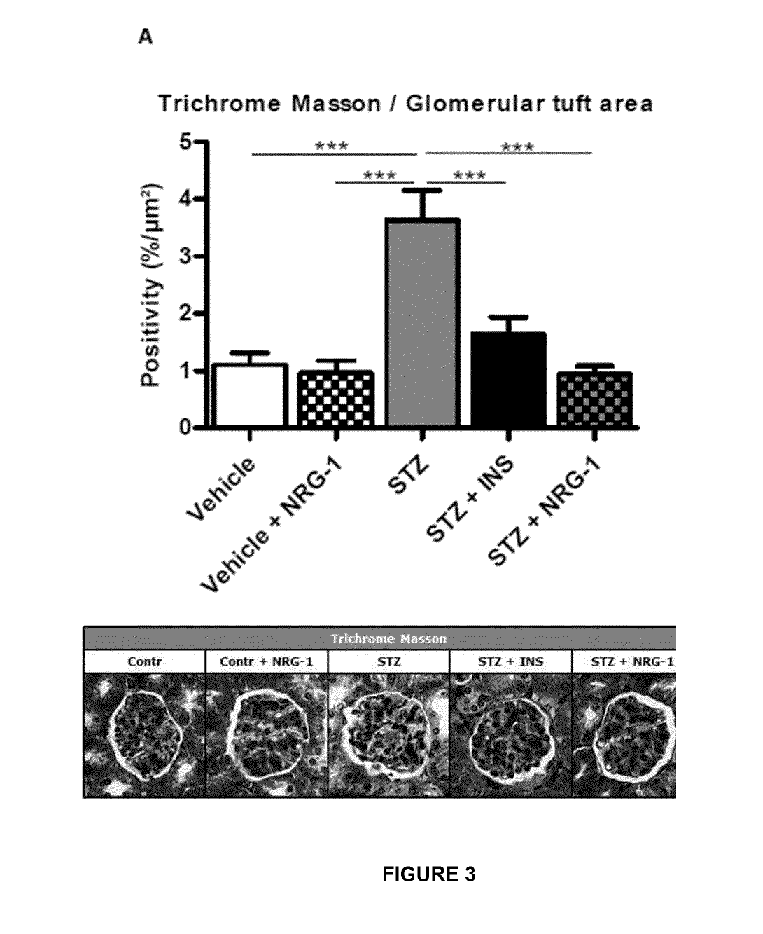

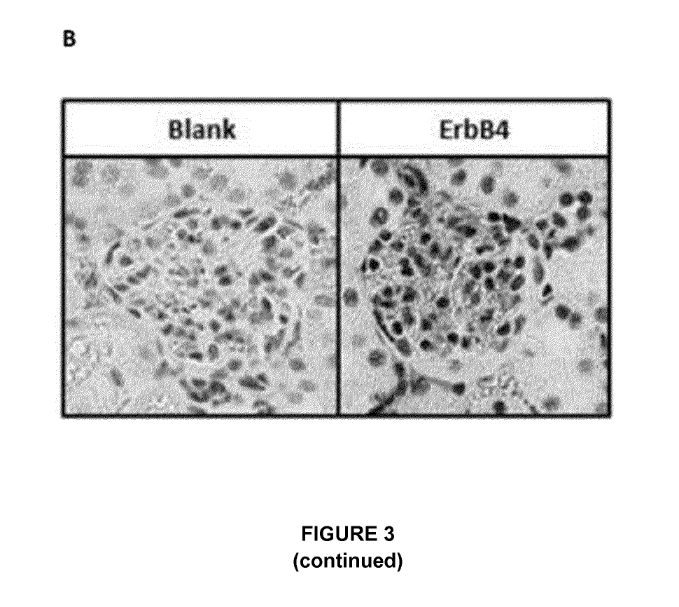

Diabetic nephropathy is characterized by gradual scarring of the glomeruli, caused by accumulation of extracellular matrix proteins (ECM) (9) which occurs early in the progression of incipient to overt nephropathy (10). Histological staining with Masson's trichrome, which stains the collagen-rich fibrotic regions, showed the presence of glomerulosclerosis in untreated diabetic animals (1.10±0.208 vs 3.63±0.504%/μm², p<0.001). Insulin treatment, as well as NRG-1 treatment, prevented glomerular scarring completely (3.63±0.504 vs 1.64±0.298%/μm², p<0.001 and 3.63±0.504 vs 0.936±0.146%/μm², p<0.001) (FIG. 3A). Immunohistological staining of kidneys of ApoE control mice showed the presence of ErbB4 receptors in the renal glomeruli (FIG. 3B). Kidney weight/tibia length ratio did not differ among the five groups (Data not shown).

Activation of Downstream Pathways

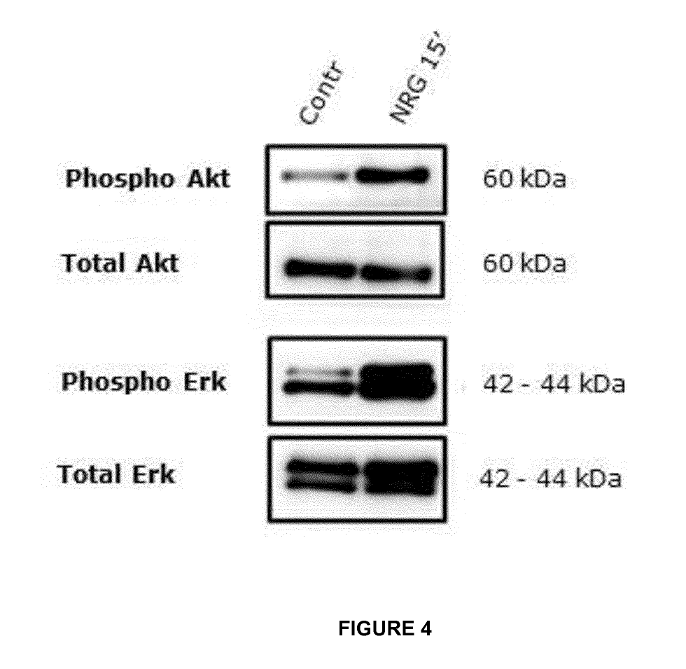

Erk and Akt pathways function downstream of neuregulin. Stimulation of mesangial cells with NRG-1 lead to phosphorylation of Erk and AKT, and thus to activation of the respective pathways, as shown in FIG. 4.

RNA Expression of Fibrotic Markers

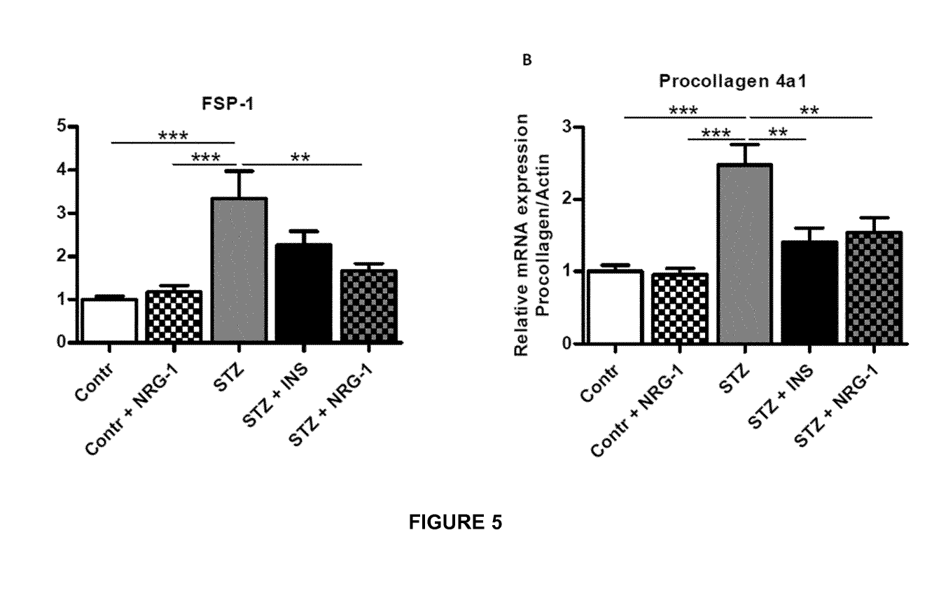

Collagen type IV and FSP-1 are markers for renal fibrosis. As shown in FIG. 5, renal expression of both FSP-1 and Collagen type IV RNA synthesis was significantly increased untreated diabetes (1.00±0.0798 vs 3.34±0.630, p<0.001 and 1.00±0.085 vs 2.47±0.283, p<0.001, respectively) in comparison with control mice. NRG-1, as well as insulin completely prevented upregulation of both fibrosis markers (3.34±0.630 vs 1.662±0.167, p<0.01 and 2.47±0.283 vs 1.54±0.206 p<0.01, respectively).

Renal Glomerular Mesangial Cells

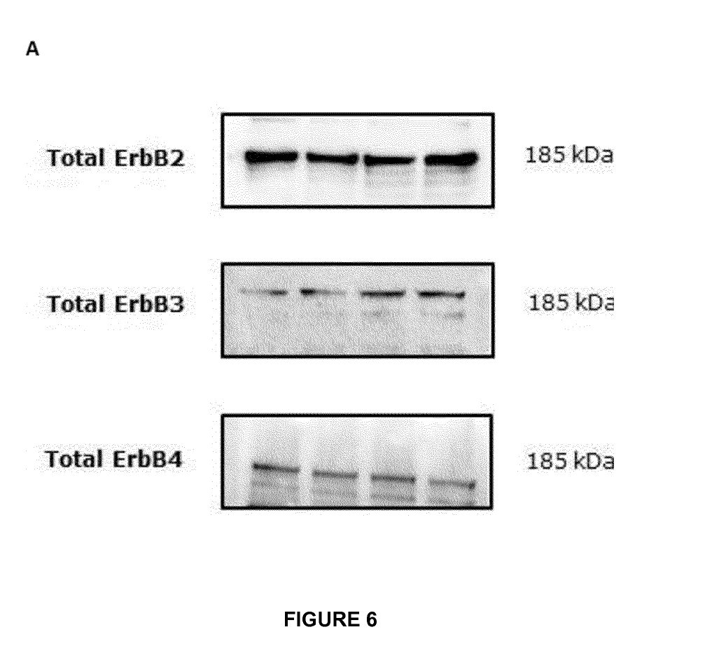

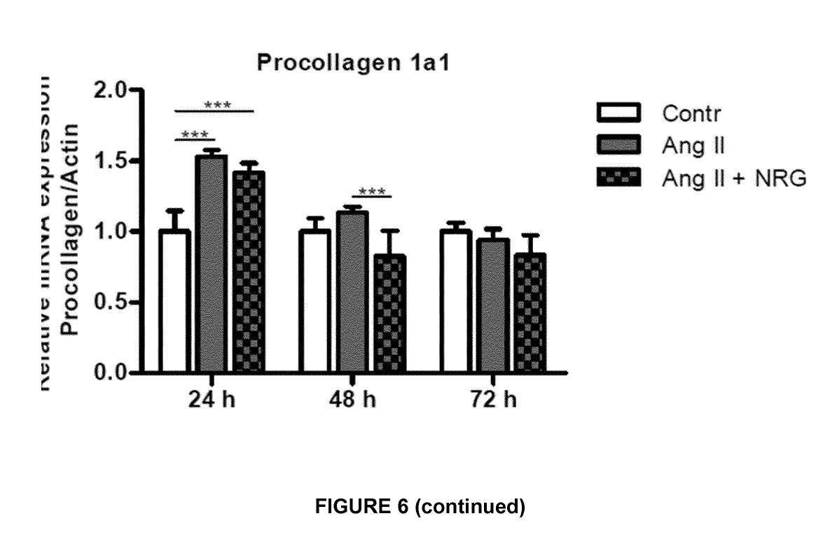

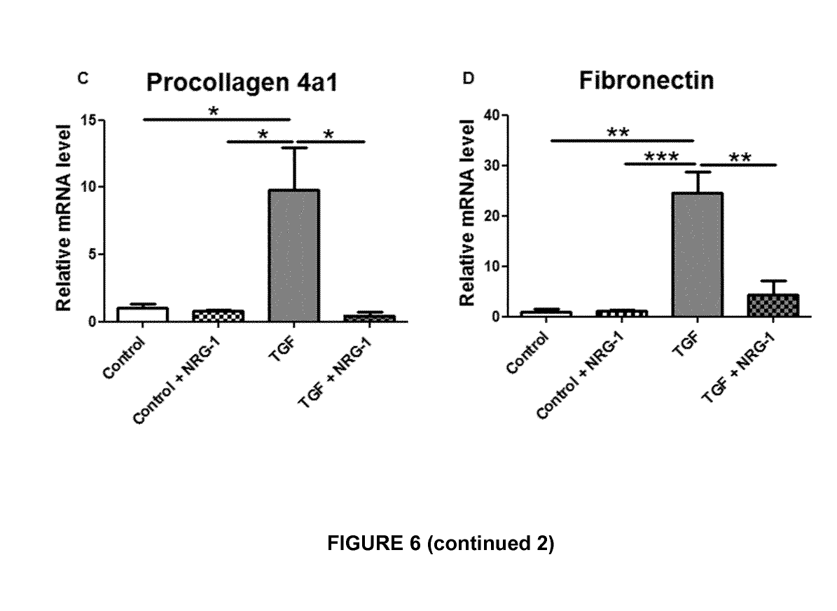

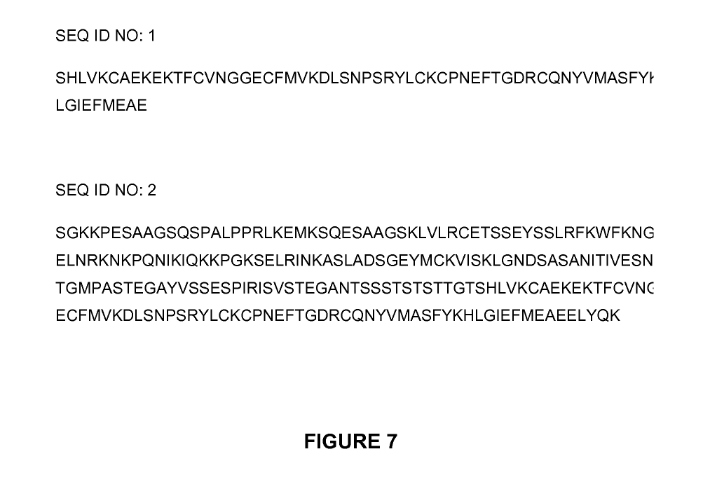

In order to reveal possible mechanisms by which NRG-1 protects the kidney, glomerular mesangial cells were studied. Mesangial cells are responsible for the synthesis of ECM proteins (11, 12) and hence play a role in the development of glomerulosclerosis. FIG. 5 shows that NRG-1 receptors, ErbB2, ErbB3 and ErbB4 are expressed by these cells (FIG. 6A). Collagen 1a1 synthesis in mesangial cells was stimulated by Ang II, and was significantly inhibited by NRG-1 over a time period of 48 hours (1.14±0.0390 vs 0.824±0.181, p<0.001) (FIG. 6B).

This and the above experiments show here for the first time, that nephropathy, in particular diabetic nephropathy, can be prevented by NRG-1 treatment. NRG-1 does not influence glucose levels indicating a direct effect on the kidney. In vivo, it has been shown here that NRG-1 prevents renal glomerular basement membrane injury (decreased albuminuria), tubular injury (decreased urine levels of NGAL) and glomerulosclerosis. In vitro, it has been shown here that NRG-1 activates the ErbB receptors present in glomerular mesangial cells. Furthermore, angiotensin II-induced collagen synthesis in mesangial cells is attenuated by NRG-1, implying an important role for the interaction between mesangial cells and NRG-1 in the course of renal fibrosis. These findings support a role for NRG-1 as a therapeutic agent for the prevention, treatment, and/or delay of nephropathy; such as diabetic nephropathy, the leading cause of end stage renal disease.

### Example 2

### Prevention of Contrast Induced Nephropathy

The effect of neuregulin on contrast induced nephropathy is demonstrated in a mouse model of contrast induced nephropathy as described below.

Animals

9 weeks old male C57Bl/6 mice were purchased from Charles River. Animals were maintained under standard laboratory conditions, 12 hour light-dark cycles with access to normal chow and drinking water at libitum. All experiments performed are approved by the ethical committee of animals (ECD) of the university of Antwerp conform to the Guide for the Care and Use of Laboratory Animals published by the US National Institutes of Health (NIH Publication No. 85-23, revised 1996).

Contrast-Induced Nephropathy (CIN) Model

Acute nephropathy was induced in mice by intraperitoneal (i.p.) injection of contrast media. To sensitize the kidney to contrast agents, mice first underwent water deprivation and pretreatment with indomethacin and L-NAME (NG-nitro-L-arginine methyl ester). Practically, following overnight water restriction, 10 animals received an injection with indomethacin (10 mg/kg, DMSO, i.p., Sigma Aldrich) and L-NAME (10 mg/kg, i.p., Sigma Aldrich) 1 hour before injection with contrast media (3 mg Iodine/kg, Visipaque). These 10 mice were divided in 2 groups: an untreated CIN group (n=5) and a CIN group treated with NRG-1 (n=5, 20 μg/kg, i.p.). 10 C57Bl/6 mice treated with buffer solution served as control animals. 5 control mice received no treatment, and 5 were treated with NRG-1 in 2 injections: 24 and 2 hours before exposure to contrast media or buffer solution. 24 hours after induction of CIN, mice were sacrificed and blood and kidneys were collected for further analysis.

Unilateral Ureteral Obstruction (UUO)

12 C57Bl/6 mice underwent ureteral obstruction of the left kidney for 7 days. Briefly, mice were anaesthetized with a mixture of ketamine and xylazine (100 mg/kg, 10 mg/kg respectively). A left abdominal incision was made and the ureter was isolated. The ureter was ligated at 2 points and cut in between, to make sure that the obstruction remained permanent. 6 mice remained untreated and 6 were daily treated with NRG-1 (20 μg/kg, i.p.) starting 24 hours before the UUO procedure. 6 days after the ligation, animals were put in metabolic cages for 24 hours for the collection of urine and were sacrificed 7 days post UUO. Blood and both of the kidneys were harvested. The obstructed kidney was analyzed for inflammation and fibrosis, while the contralateral kidney of each animal served as a control. For the measurement of kidney function markers, 2 groups of animals, untreated (n=5) or treated with NRG-1 (n=5) were added which underwent a sham operation without obstructing the left kidney ureter, serving as control animals.

Kidney Function Markers

After centrifugation of the blood samples, plasma was isolated. Both urinary and plasma creatinine were measured to determine creatinine clearance. Creatinine was analyzed by an enzymatic reaction via autoanalyzer (Siemens Vista 1500).

Histological Assessment of Renal Injury

Left kidneys of the CIN model and left and right kidneys of the UUO model were weighted and partly fixed in 4% buffered formalin and embedded in paraffin for histological staining according to standard procedures. Kidney sections (5 μm) were stained with periodic-acid schiff base (PAS) to evaluate the general renal morphology, Sirius Red in order to determine the degree of glomerulosclerosis, 3,3′-diaminobenzidine (DAB) substrate kit for the analysis of neutrophil infiltration and Mac-3 (M3/84, Santa Cruz) for the staining of inflammatory cells.

RNA Extraction and Real-Time PCR

Right kidneys from the CIN-mice and half of the right and left kidney from the UUO-animals were snap-frozen in liquid nitrogen at sacrifice. Kidney tissue was then homogenized using a Polytron homogenizer (Pt 2100; Kinematica, Littau, Switzerland) and RNA was obtained by the GenElute Mammalian Total RNA Miniprep Kit (Sigma Aldrich). Total RNA was transcribed to cDNA using random hexamers (TaqMan Reverse Transcription Reagents, Applied Biosystems). Using TaqMan real-time PCR (Life Technologies), procollagen 1a1 (Col Ia1, Mm00801666_g1), procollagen 3a1 (Col IIIa1, Mm01254476_m1), fibronectin-1 (Mm01256744_m1), Transforming Growth Factor-β1, (TGF-β1, Mm01178820_m1), Intercellulaire Adhesie Molecule-1 (ICAM-1, Mm00516023_m1) and Vasculair Cel Adhesie Molecule-1 (VCAM-1, Mm01320970_m1) mRNA expression was analyzed against β-actine (Mm00607939_s1, Life Technologies) in whole kidney tissue.

Data Analysis and Statistics

Data are expressed as means±SEM. Differences between groups were analyzed by one-way ANOVA with Bonferroni corrections for multiple comparisons. Western blots were subjected to densitometric analysis using ImageJ 1.42 software. Statistical significance was defined as P<0.05. All statistical analyses were done using Graph Pad Prism 5 and IBM SPSS Statistics 22 software.

Results

Contrast-Induced Nephropathy

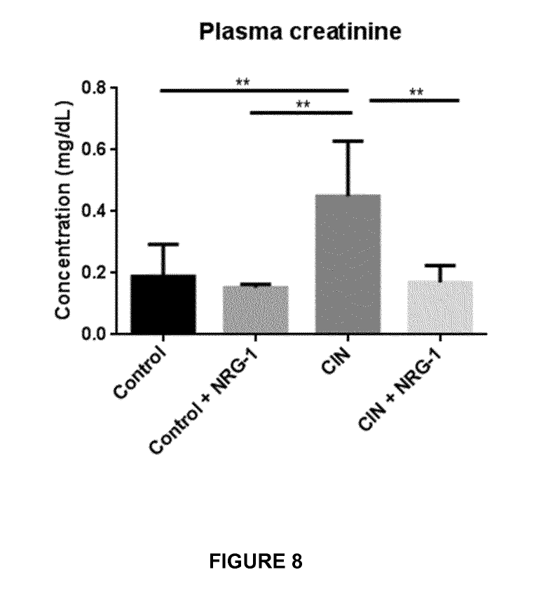

There was no difference in kidney weight among the 4 groups (data not shown). Measurement of plasma creatinine 24 hours after injection with contrast media showed a significant increase in the untreated CIN-group. Accumulation of creatinine in plasma is an indication of a reduced glomerular filtration rate of the kidney. Treatment of these animals with NRG-1 prevented plasma creatinine accumulation, hence protected the kidney against contrast-induced kidney failure, as shown in FIG. 8.

These experiments demonstrate that NRG-1 protects against acute contrast-induced renal failure and hence may be applied to prevent this failure.

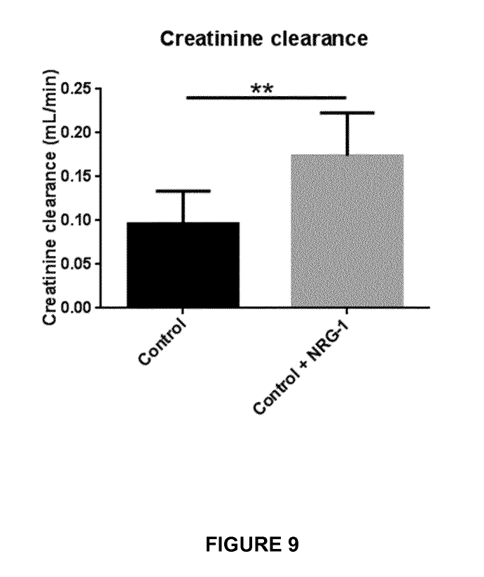

In the previous experiments, the creatinine clearance of the contrast-treated animals could not be determined, since the animals stop producing urine after contrast injection. However, the control group in these experiments (prehydrated during 24 hours) does produce urine, hence allowing us to calculate the creatinine clearance of these mice. Interestingly, as shown in FIG. 9, NRG-1 increases creatinine clearance in these conditions.

This is a very important experimental result, since it indicates that NRG-1, in conditions of an activated renal angiotensin-system (here triggered by water deprivation) increases glomerular filtration rate. This is clinically very important since this indicates that NRG-1 is useful as a drug to increase glomerular filtration rate (GFR). This may be useful in conditions of severe acute kidney injury, or in conditions of chronic kidney failure to delay hemodialysis. To date, there are no drugs available to increase GFR.

Ureteral Ligation-Induced Renal Fibrosis

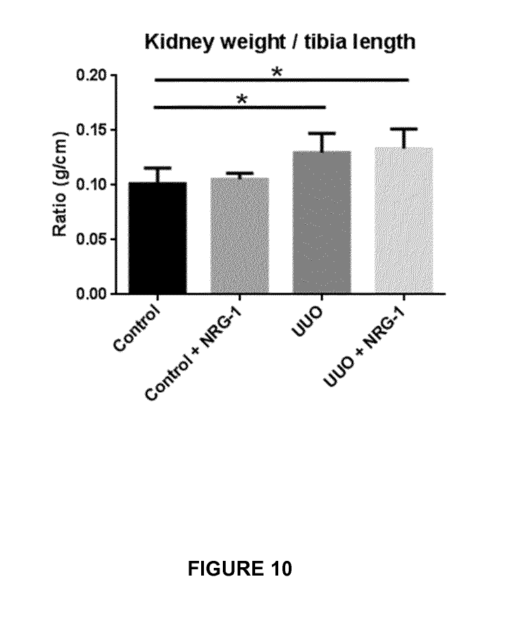

The weight of the obstructed kidney was significantly higher than the contralateral control kidney, independently of NRG-1 treatment (FIG. 10).

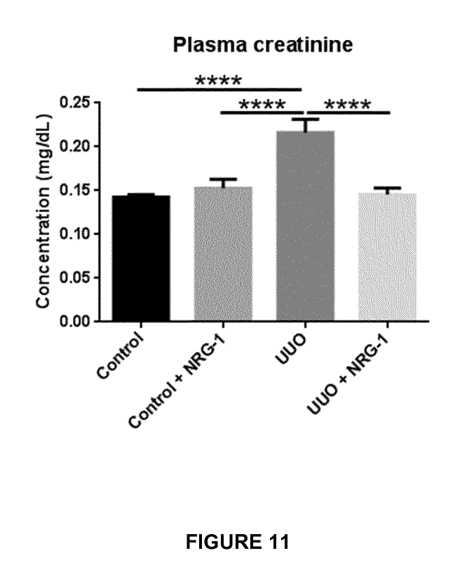

FIG. 11 shows that plasma creatinine is increased in UUO, indicating that glomerular kidney function was decreased. NRG-1 significantly prevented this increase.

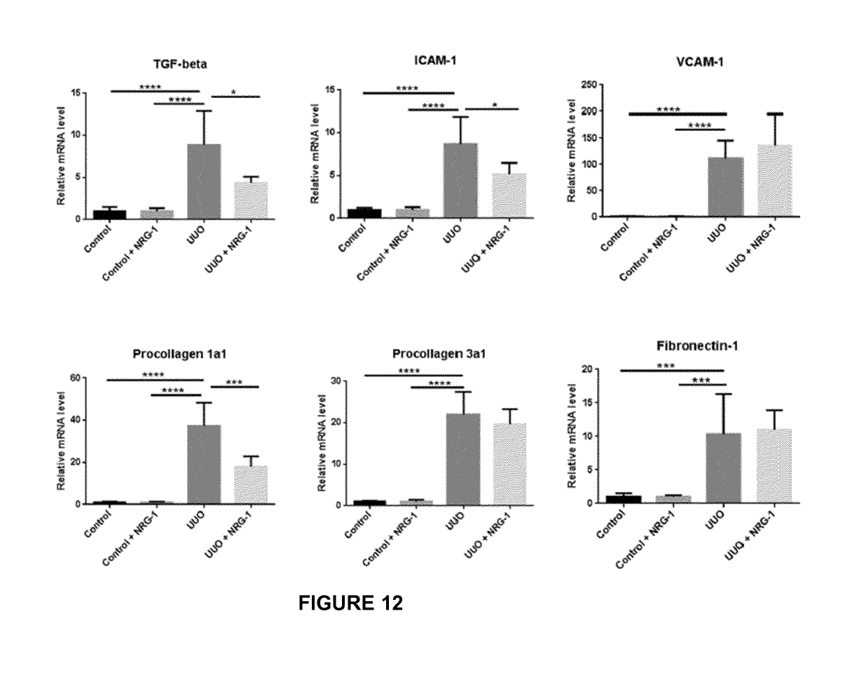

Analysis of inflammation markers on mRNA level showed how TGF-beta, ICAM-1 and VCAM-1 are significantly upregulated in the renal ligation kidneys in comparison with the contralateral kidneys. Also fibrotic markers, including procollagen 1a1, 3a1 and fibronectin mRNA were significantly higher in the UUO kidneys. Treatment with NRG-1 has no effect on control kidneys, but remarkably reduced TGF-beta, ICAM-1 and procollagen 3a1 in the obstructed kidneys (FIG. 12).

This demonstrates that NRG-1 protects against “acute” ureter-ligation-induced renal inflammation and fibrosis.

### REFERENCES

(1) U.S. Renal Data System, USRDS 2012. Annual data report: atlas of chronic kidney disease and end-stage renal disease in the United States. National Institutes of Health, National Institute of Diabetes and Digestive and Kidney Diseases, Bethesda, Md. Accessed 6 Jan. 2013
(2) P H Groop et al., The Presence and Severity of Chronic Kidney Disease Predicts All-Cause Mortality in Type 1 Diabetes, Diabetes, 58 (7): 1651-1658 (2009)
(3) M Afkarian et al., Kidney Disease and Increased Mortality Risk in Type 2 Diabetes, JASN, 24 (2): 302-308 (2013)
(4) T J Orchard, A M Secrest, R G Miller, T Costacou, In the absence of renal disease, 20 year mortality risk in type 1 diabetes is comparable to that of the general population: a report from the Pittsburgh Epidemiology of Diabetes Complications Study, Diabetologia, 53(11): 2312-2319 (2010)
(5) I H de Boer et al., Temporal trends in the prevalence of diabetic kidney disease in the United States, JAMA, 305: 2532-2539 (2011)
(6) E T Rosolowsky et al., Risk for ESRD in type 1 diabetes remains high despite renoprotection, J Am Soc Nephrol, 22: 545-553 (2011)
(7) G P Rangan, G H Tesch, Methods in Renal Research, Quantification of Renal Pathology by Image Analysis, Nephrology, 12: 553-558 (2007)
(8) Lemmens K, Doggen K, De Keulenaer G W., Activation of the neuregulin/erbb system during physiological ventricular remodeling in pregnancy. American Journal of Physiology-Heart and Circulatory Physiology, 300:H931-H942 (2011)
(9) Y Qian, E Feldman, S Pennathur, M Kretzler, F C Brosius, From Fibrosis to Sclerosis, Mechanisms of Glomerulosclerosis in Diabetic Nephropathy, Diabetes, 57: 1439-1445 (2008)
(10) M S Simonson, Phenotypic transitions and fibrosis in diabetic nephropathy, Kidney International, 71: 846-854 (2007)
(11) M A Haralson, Collagen polymorphism in cultured rat kidney mesangial cells, Lab Invest 57: 513-523 (1987)
(12) N Ardaillou, G Bellon, M-P Nivez, S Rakotoarison, R Ardaillou, Quantification of collagen synthesis by cultured human glomerular cells, Biochem Biophys Res Comm, 991: 445-452 (1989)
(13) Khan A M, Maderdrut J L, Li M, Toliver H L, Coy D H, Simon E E, Batuman V. Pituitary adenylate cyclase-activating polypeptide prevents contrast-induced nephropathy in a novel mouse model. Physiol Rep. 2013 November; 1(6):e00163. doi: 10.1002/phy2.163. Epub 2013 Nov. 19.
(14) Kodama A, Watanabe H, Tanaka R, Tanaka H, Chuang V T, Miyamoto Y, Wu Q, Endo M, Hamasaki K, Ishima Y, Fukagawa M, Otagiri M, Maruyama T. A human serum albumin-thioredoxin fusion protein prevents experimental contrast-induced nephropathy. Kidney Int. 2013 March; 83(3):446-54. doi: 10.1038/ki.2012.429. Epub 2013 January

1-16. (canceled)

17. A method of treating, preventing or delaying nephropathy in a mammal, said method comprising administering to a mammal at risk or suffering from nephropathy a neuregulin (NRG) protein or a functional fragment thereof, or a nucleic acid encoding said NRG protein or said functional fragment of an NRG protein, thereby treating, preventing or delaying nephropathy in said mammal.

18. The method according to claim 1, wherein said nephropathy is acute tubular necrosis (ATN).

19. The method according to claim 1, wherein said nephropathy is chronic nephropathy.

20. The method according to claim 1, wherein said nephropathy is acute nephropathy

21. The method according to claim 1, wherein said nephropathy is selected from the group consisting of diabetic nephropathy (nephrangiosclerosis), nephropathy caused by hypertension, nephropathy caused by vasculitis, lupus nephritis, nephropathy caused by glomerulonephritis, nephropathy caused by tubuloinsterstitial diseases, obstructive nephropathy, contrast induced nephropathy, toxic nephropathy, glomerulonephritis, and acute interstitial nephritis (AIN).

22. The method according to claim 1, wherein said nephropathy is characterized by one or more symptoms selected from albuminuria, glomerulosclerosis, and/or renal fibrosis.

23. The method according to claim 1, wherein said NRG protein is a neuregulin-1 (NRG-1) protein, a neuregulin-2 (NRG-2) protein, a neuregulin-3 (NRG-3) protein, a neuregulin-4 (NRG-4) protein, or mixtures thereof.

24. The method according to claim 1, wherein said functional fragment comprises an EGF-like domain.

25. The method according to claim 1, wherein said NRG protein is an NRG-1 protein.

26. The method according to claim 1, wherein said NRG protein is a bivalent NRG protein.

27. The method according to claim 1, wherein said NRG protein is administered daily.

28. The method according to claim 1, wherein said NRG protein is administered in a daily dose ranging from 0.01 to 100 μg/kg body weight.

29. The method according to claim 1, wherein said mammal is a human.
